(19) United States
(12) Patent Application Publication
(10) Pub. No.: US 2017/0199176 A1
(43) Pub. Date: Jul. 13, 2017

(54) T CELL POPULATIONS IN DIAGNOSIS, PROGNOSIS, PREDICTION, AND MONITORING

(71) Applicant: Nodality, Inc., South San Francisco, CA (US)

(72) Inventors: Andrew Hotson, Redwood City, CA (US); Rachael Hawtin, San Carlos, CA (US)

(21) Appl. No.: 15/403,026

(22) Filed: Jan. 10, 2017

Related U.S. Application Data
(60) Provisional application No. 62/294,232, filed on Feb. 11, 2016.
(60) Provisional application No. 62/277,375, filed on Jan. 11, 2016.

(51) Int. Cl. G01N 33/50

(52) U.S. Cl. CPC G01N 33/5091; G01N 33/5023; G01N 2800/52

(57) ABSTRACT

Disclosed herein are methods and compositions for determining the proclivity of immune cells to polarize to one or more immune cell subpopulations (e.g., Th17 population) and/or to produce one or more intracellular substances (e.g., after stimulation).

### CROSS-REFERENCE

This application claims the benefit of U.S. Provisional Patent Application No. 62/277,375, filed on Jan. 11, 2016 and U.S. Provisional Patent Application No. 62/294,232, filed on Feb. 11, 2016, each of which is entirely incorporated herein by reference.

### SUMMARY OF THE INVENTION

In one aspect, the invention provides methods.

In certain embodiments, the invention provides a method comprising (i) contacting immune cells from a sample with one or more activators of T cells; (ii) incubating the cells for less than 6 days; and (iii) determining the frequency of Th17 cells in the sample after the incubation. The cells can be incubated, for example, between 4 and 5.8 days. The cells can be incubated, for example, between 4.5 and 5.5 days. The cells can be incubated, for example, for 5 days. Alternatively or additionally, the frequency of another Tcell subpopulation is determined, such as a Th1 subpopulation. The method can further comprise treating the cells with a modulator of a non-T cell population, such as an activator of a non-T cell population, for example a toll-like receptor (TLR) activator, e.g., an activator of TLR4 or an activator of TLR7/8. In certain embodiments, the TLR activator comprises an activator of TLR4 comprising LPS. In certain embodiments, the TLR activator comprises an activator of TLR7/8 comprising R848. The method can further comprise determining cell health before determining frequency of Th17 cells, and eliminating cells that are not healthy, for example, by determining the level of a marker of apoptosis, for example c-PARP, and, e.g., eliminating cells whose level of the marker is above a threshold level. The method can further comprise contacting the immune cells with an inhibitor of one or more T cell subpopulations, such as an inhibitor of Th2 cell subpopulations and/or an inhibitor of Th1 cell subpopulations. Exemplary inhibitors include anti-IL4, anti-IFNg, or a combination thereof. In certain embodiments of the method, the frequency of Th17 cells is determined by determining intracellular levels of one or more markers in single cells, such as IL-17A, IL-17F, or IL-17AF, or a combination thereof. The determination of IL-17 levels can be performed, e.g., by flow cytometry or mass cytometry. For the determination, the cells can be contacted with detectable binding agents, e.g., antibodies, specific for IL-17 (and other markers to be determined, e.g., cytokines, activatable elements, and/or cell surface markers). In certain embodiments, the immune cells are from a blood sample or a blood-derived sample, or a tumor infiltrating lymphocyte (TILS) or TILS-derived sample. For example, the sample can be a peripheral blood mononuclear cell sample. In certain embodiments the method further comprises treating the cells with a cytokine, for example, TNFa, TGFb, IL-1b, IL-21, I1-6 or IL-23, or a combination thereof. In certain embodiments, the cytokine comprises IL-6 or IL-23 or a combination thereof. In certain embodiments of the method, the levels of TNFa and/or IFNg are also determined in single cells. In certain embodiments, the method further comprises determining the level of one or more cell surface markers on the single cells, for example, CD3, CD4, CD8, or a combination thereof. In certain embodiments, the method further comprises determining the level of IL-21, IL-22, or both, in the cells.

In certain embodiments, the invention provides a method comprising (i) contacting immune cells in a culture derived from a sample with one or more activators of T cells and one or more modulators of a non-T cell population; (ii) incubating the cells; (iii) determining the frequency of Th17 cells in the sample after the incubation. In certain embodiments, the modulator comprises an activator of a non-T cell population, for example a toll-like receptor (TLR) activator, e.g., an activator of TLR4 or an activator of TLR7/8. In certain embodiments, the TLR activator comprises an activator of TLR4 comprising LPS. In certain embodiments, the TLR activator comprises an activator of TLR7/8 comprising R848. In certain embodiments, the incubation time is less than 8 days, for example, less than 6 days, such as between 3 and 5.8 days, or between 4 and 5.8 days, or between 4.5 and 5.5 days, or about 5 days. The method can further comprise determining cell health before determining frequency of Th17 cells, and eliminating cells that are not healthy, for example, by determining the level of a marker of apoptosis, for example c-PARP, and, e.g., eliminating cells whose level of the marker is above a threshold level. The method can further comprise contacting the immune cells with an inhibitor of one or more T cell subpopulations, such as an inhibitor of Th2 cell subpopulations and/or an inhibitor of Th1 cell subpopulations. Exemplary inhibitors include anti-IL4, anti-IFNg, or a combination thereof. In certain embodiments of the method, the frequency of Th17 cells is determined by determining intracellular levels of one or more markers in single cells, such as IL-17A, IL-17F, or IL-17AF, or a combination thereof. The determination of IL-17 levels can be performed, e.g., by flow cytometry or mass cytometry. For the determination, the cells can be contacted with detectable binding agents, e.g., antibodies, specific for IL-17 (and other markers to be determined, e.g., cytokines, activatable elements, and/or cell surface markers). In certain embodiments, the immune cells are from a blood sample or a blood-derived sample, or a tumor infiltrating lymphocyte (TILS) or TILS-derived sample. For example, the sample can be a peripheral blood mononuclear cell sample. In certain embodiments the method further comprises treating the cells with a cytokine, for example, TNFa, TGFb, IL-1b, IL-21, 11-6 or IL-23, or a combination thereof. In certain embodiments, the cytokine comprises IL-6 or IL-23 or a combination thereof. In certain embodiments of the method, the levels of TNFa and/or IFNg are also determined in single cells. In certain embodiments, the method further comprises determining the level of one or more cell surface markers on the single cells, for example, CD3, CD4, CD8, or a combination thereof. In certain embodiments, the method further comprises determining the level of IL-21, IL-22, or both, in the cells.

In certain embodiments, the invention provides a method comprising (i) contacting immune cells in a culture derived from a sample with one or more activators of T cells; (ii) incubating the cells; (iii) determining the level of cell health for single cells of the immune cells and eliminating unhealthy cells from analysis; (iv) determining the frequency of Th17 cells in the sample after the incubation and after eliminating unhealthy cells. Cell health can be determined, for example, by determining the level of a marker of apoptosis, for example c-PARP, and, e.g., eliminating cells whose level of the marker is above a threshold level. In certain embodiments, the method can further comprise contacting the cells with a modulator of a non-T cell population. In certain embodiments, the modulator comprises an activator of a non-T cell population, for example a toll-like receptor (TLR) activator, e.g., an activator of TLR4 or an activator of TLR7/8. In certain embodiments, the TLR activator comprises an activator of TLR4 comprising LPS. In certain embodiments, the TLR activator comprises an activator of TLR7/8 comprising R848. In certain embodiments, the incubation time is less than 8 days, for example, less than 6 days, such as between 3 and 5.8 days, or between 4 and 5.8 days, or between 4.5 and 5.5 days, or about 5 days. The method can further comprise contacting the immune cells with an inhibitor of one or more T cell subpopulations, such as an inhibitor of Th2 cell subpopulations and/or an inhibitor of Th1 cell subpopulations. Exemplary inhibitors include anti-IL4, anti-IFNg, or a combination thereof. In certain embodiments of the method, the frequency of Th17 cells is determined by determining intracellular levels of one or more markers in single cells, such as IL-17A, IL-17F, or IL-17AF, or a combination thereof. The determination of IL-17 levels can be performed, e.g., by flow cytometry or mass cytometry. For the determination, the cells can be contacted with detectable binding agents, e.g., antibodies, specific for IL-17 (and other markers to be determined, e.g., cytokines, activatable elements, and/or cell surface markers). In certain embodiments, the immune cells are from a blood sample or a blood-derived sample, or a tumor infiltrating lymphocyte (TILS) or TILS-derived sample. For example, the sample can be a peripheral blood mononuclear cell sample. In certain embodiments the method further comprises treating the cells with a cytokine, for example, TNFa, TGFb, IL-1b, IL-21, 11-6 or IL-23, or a combination thereof. In certain embodiments, the cytokine comprises IL-6 or IL-23 or a combination thereof. In certain embodiments of the method, the levels of TNFa and/or IFNg are also determined in single cells. In certain embodiments, the method further comprises determining the level of one or more cell surface markers on the single cells, for example, CD3, CD4, CD8, or a combination thereof. In certain embodiments, the method further comprises determining the level of IL-21, IL-22, or both, in the cells.

In certain embodiments, the invention provides a method of monitoring an aspect of a condition in an individual, comprising (i) contacting a sample from the individual comprising immune cells with an activator of differentiation of Th17 cells; (ii) incubating the cells for a period of time; (iii) determining the change in frequency of Th17 cells in the sample [or determining levels of IL-17 in the cells]; and (iv) from the results of (iii), determining a characteristic of the aspect of the condition in the individual. In certain embodiments of the method, the individual suffers from an autoimmune condition, such as multiple sclerosis, systemic lupus erythematosus, or rheumatoid arthritis, for example, rheumatoid arthritis (RA). In certain embodiments of the method, the individual suffers from cancer, such as melanoma, non-small cell lung carcinoma, small cell lung cancer, bladder cancer, or prostate cancer; for example, melanoma. In certain embodiments, the aspect of the condition is treatment of the condition. For example, the condition can be cancer and the treatment is treatment for the cancer, for example an immunomodulatory treatment, such as treatment with a checkpoint inhibitor, for example ipilimumab. In certain embodiments of the method, the characteristic of the treatment comprises development of adverse effect; progression, regression, or stasis of the condition; response to a treatment; or a combination thereof. In certain embodiments, the characteristic comprises development of adverse effect, for example, development of colitis. In certain embodiments, the condition is an autoimmune condition and the treatment is a treatment for the autoimmune condition. Non-limiting examples of autoimmune conditions include multiple sclerosis, systemic lupus erythematosus, or rheumatoid arthritis (RA). In certain embodiments, the autoimmune condition is RA.

In certain embodiments, the invention provides a method of predicting development of colitis in an individual receiving treatment comprising administration of ipilimumab or potentially comprising administration of ipilimumab comprising (i) contacting immune cells from a sample from the individual with an activator of Th17 cell differentiation; (ii) incubating the cells for a period of time; (iii) determining the change in frequency of Th17 cells after the incubation; and (iv) from the results of (iii), determining whether or not, or the likelihood, that the individual will develop colitis if the administration of ipilimumab is continued or undertaken. In certain embodiments, the individual is receiving ipilimumab and the method further comprises modifying the treatment of the individual based at least in part on the determination of (iv). The modification can include, e.g., modifying the dose of ipilimumab, modifying the schedule of dosing of ipilimumab, discontinuing ipilimumab, adding an agent to the treatment, or a combination thereof. In certain embodiments, the individual is potentially receiving ipilimumab and the method comprises administering or not administering ipilimumab based at least in part on the determination of (iv). In certain embodiments, the individual suffers from cancer, such as melanoma, small cell lung cancer, non-small cell lung carcinoma, bladder cancer, or prostate cancer, for example, melanoma. In certain embodiments, the method alternatively or additionally includes determining the levels of one or more intracellular activatable elements in immune cells in the sample, such as when the cells have further been contacted with a modulator that is not an activator of Th17 cell differentiation.

In certain embodiments, the invention provides a method of predicting development of colitis in an individual receiving treatment comprising administration of ipilimumab or potentially comprising administration of ipilimumab comprising (i) determining the levels of one or more activatable elements in immune cells in a sample from the individual; and (ii) from the results of (i), determining whether or not, or the likelihood, that the individual will develop colitis if the administration of ipilimumab is continued or undertaken. In certain embodiments, the method can further comprise contacting the cells with a modulator. The activatable activatable element can be, e.g., p-Stat 1, p-Stat 3, p-Stat 6, p-p38, p-ERK, IKba, p-S6, p-TBK, p-AKT, IKBa, or a combination thereof. When a modulator is used, it can be, e.g., IL-6, IL-23, R848, IL-1b, LPS, or a combination thereof. In certain embodiments, the modulator comprises IL-6, IL-23, or a combination thereof, and the activatable element comprises p-Stat 1, p-Stat 3, p-Stat 6, or a combination thereof. In certain embodiments, the modulator comprises R848, IL-1b, or a combination thereof, and the activatable element comprises p-p38, p-ERK, IKba, p-S6, p-TBK, p-AKT, or a combination thereof. In certain embodiments, the modulator comprises LPS and the activatable element comprises p-p38, IKba, p-S6, or a combination thereof.

In certain embodiments, the invention provides a method of screening agents comprising (i) contacting immune cells with an activator of Th17 differentiation; (ii) contacting the cells with an agent; (iii) incubating the cells for a period of time; and (iv) determining the frequency of Th17 cells after the incubation. The method can further comprise determining whether or not to advance the agent to a further level of screening or tests based at least in part on the results of (iv). The agents can be screened for potential efficacy in treating one or more conditions. The agents can be screened for potential adverse effects. In certain embodiments, the condition comprises an autoimmune condition, such as multiple sclerosis, systemic lupus erythematosus, or rheumatoid arthritis (RA), for example, RA. In certain embodiments, the agent is an anti-cytokine agent, e.g., an anti-cytokine antibody.

In one aspect, the invention provides compositions.

In certain embodiments, the invention provides a kit comprising (i) an activator of T cells; (ii) a modulator of a non-T cell population; (iii) a detectable binding element, such as an antibody, specific for IL-17; (iv) an agent for inducing IL-17 formation. The modulator of a non-T cell population can comprise an activator of a non-T cell population, for example, a toll-like receptor (TLR) activator, such as a TLR4 activator, e.g., LPS, and/or a TLR7/8 activator, e.g., R848. The kit can further comprise one or more detectable binding elements to a cell surface marker, for example, a cell surface marker selected from the group consisting of CD3, CD4, CD8, and combinations thereof. In certain embodiments, the activator of T cells is a TCR activator, such as anti-CD3, anti-CD28, or a combination thereof. The kit can further comprise a detectable binding element for a marker of cell health, such as a marker of apoptosis, e.g., cPARP. The kit can further comprise instructions. The kit can further comprise packaging to hold components of the kit.

In certain embodiments the invention provides a kit comprising (i) an activator of T cells; (ii) a detectable binding element for a marker of apoptosis, for example, c-PARP; (iii) a detectable binding element specific for IL-17; (iv) an agent for inducing IL-17 formation. The kit can further comprise a modulator of a non-T cell population, The modulator of a non-T cell population can comprise an activator of a non-T cell population, for example, a toll-like receptor (TLR) activator, such as a TLR4 activator, e.g., LPS, and/or a TLR7/8 activator, e.g., R848. The kit can further comprise one or more detectable binding elements to a cell surface marker, for example, a cell surface marker selected from the group consisting of CD3, CD4, CD8, and combinations thereof. In certain embodiments, the activator of T cells is a TCR activator, such as anti-CD3, anti-CD28, or a combination thereof. The kit can further comprise instructions. The kit can further comprise packaging to hold components of the kit.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows representative receptors, modulators, and activatable elements of the invention.

FIG. 2 shows an exemplary protocol for a TH17 polarizing assay for a duration of less than 6 days, in this case, 5 days.

FIG. 3 shows that peripheral blood mononuclear cells (PBMC) samples obtained from melanoma patients prior to ipilimumab treatment and treated to induce Th17 polarization show significant association between Th17 cell number and development of Grade 3 colitis with ipilimumab treatment.

FIG. 4 shows PBMC samples obtained from melanoma patients prior to ipilimumab treatment and treated to induce Th17 polarization show significant association between Th17 cell number and colitis severity with ipilimumab treatment.

FIG. 5 shows PBMC samples obtained from melanoma patients after 6 weeks of ipilimumab treatment and treated to induce Th17 polarization show significant association between Th17 cell number and development of Grade 3 colitis with ipilimumab treatment.

FIG. 6 shows PBMC samples obtained from melanoma patients after 6 weeks of ipilimumab treatment and treated to induce Th17 polarization show significant association between Th17 cell number and colitis severity with ipilimumab treatment.

FIG. 7 shows a summary of results from FIGS. 3-6.

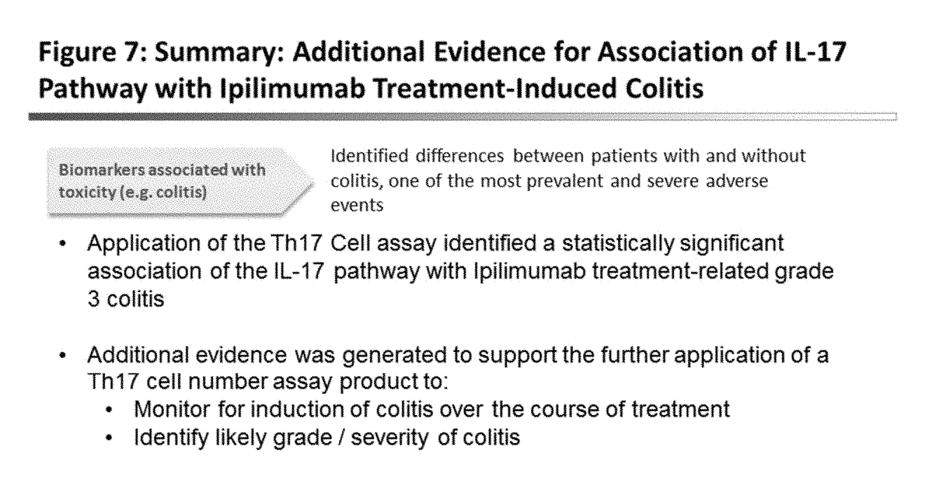

FIG. 8 shows additional parameters that can increase the predictive power of the Th17 assay product

FIG. 9 shows potential feedback mechanisms for increased or decreased Th17 cells in RA and in ipilimumab treatment-associated colitis.

### DETAILED DESCRIPTION OF THE INVENTION

In certain aspects, the invention provides methods and compositions to determine the proclivity of immune cells to polarize to one or more immune cell subpopulations and/or to produce one or more intracellular substances, e.g., cytokines, such as IL-17, TNFa, and/or IFNg, after stimulation and incubation. Typically, the cells are T cells and the subpopulation is a Th17 population (cytokine=IL-17); the subpopulation may additionally or alternatively be a Th1 (cytokine=IFNg) or Th2 cell supbpopulation. The methods and compositions may further be used to diagnose, prognose, predict, or monitor a condition or an aspect of a condition, e.g., an autoimmune condition or a cancer; for screening agents; and for any other use where proclivity of cells to differentiate to T cell subpopulations and/or to be stimulated to produce modulated levels of one or more intracellular markers determined can be used.

The cell type, e.g., immune cell subpopulation type can be determined by determining levels of one or more intracellular markers and cell surface markers in single cells in a sample. The intracellular marker can be a marker for a particular type of Th cell, such as IL-17 (Th17 cells), e.g., IL-17A, IL-17F, and/or IL-17AF; other intracellular substances may be used, such as IFN, e.g., IFNg; TNF, e.g., TNFa or TNFb, TNFa preferred; and/or one or more interleukins, such as IL-2, IL-10, to determine Th1 cells. The levels of the one or more intracellular markers can be measured on a single cell basis, e.g., by cytometry, such as by flow cytometry or mass cytometry. Cell surface markers can include markers for a cell subpopulation such as CD3, CD4, CD8, and the like, as detailed further below.

In certain cases extracellular levels of one or more markers produced after stimulation may additionally or alternatively be used, e.g., extracellular levels of IL-17. In certain cases, combined levels of markers from a plurality of cells may be used. However, in general, the methods and compositions are directed to single cells; i.e., measurements on single cells that are then typically combined to produce data of use in the methods and compositions, for example, frequency of Th17 cells.

Cells can be contacted with one or more activators, optionally also including one or more polarizing cytokines, and/or one or more substances that act to inhibit one or more polarizing cytokines; incubating the cells for a period of time, and measuring one or more characteristics of the cells and/or of the environment of the cells to determine the frequency of one or more immune cell subpopulations, e.g., the frequency of Th1, Th2, and/or Th17 subpopulation. In certain embodiments, the immune cell subpopulation comprises a Th17 subpopulation. In certain embodiments, the immune cell subpopulation comprises a Th1 subpopulation. Generally, the frequency of the subpopulation is also determined before activation and/or incubation and compared to the frequency after activation and incubation, to provide a change in frequency for the subpopulation.

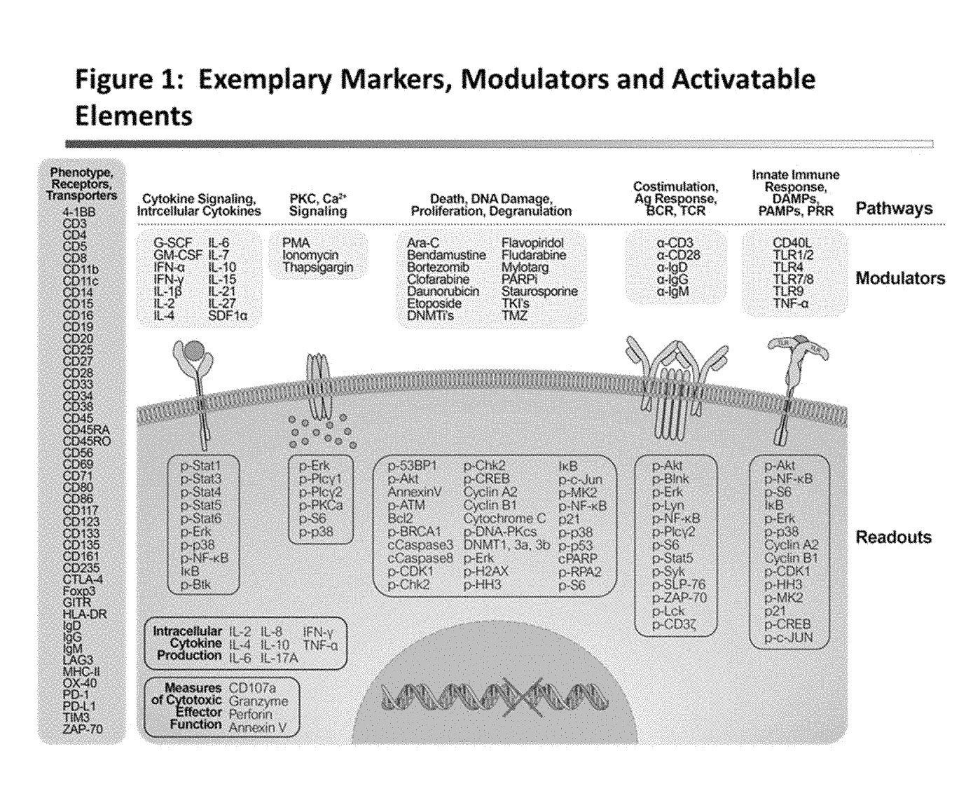

Alternatively, or additionally, levels of activatable elements in the cells may be determined, either with or without modulation of the cells with a modulator. Examples of modulators and activatable elements are shown in FIG. 1.

### Th17 Polarization Assay

T cell polarization. In one aspect, the invention provides methods and compositions to activate cells toward a Th17 phenotype; additional phenotypes, such as the Th1 and/or Th2 phenotype may also be determined. The method includes activating cells from a sample containing immune cells, such as a blood or tissue sample, using one or more T cell receptor (TCR) activators and, in certain embodiments one or more activators of non-T immune cells, such as toll-like receptor (TLR) activators. The cells are incubated for a period of time under suitable conditions. The time period may be any suitable time period, but is generally less than 6 days. The frequency of Th17 cells is then determined and, typically, compared to the frequency before incubations. The Th17 phenotype can be determined in single cells, for example, by flow or mass cytometric measurements of cell surface markers and one or more intracellular markers, for example, IL-17, such as IL-17A. In certain embodiments, unhealthy cells, e.g., cells undergoing apoptosis, are eliminated from the analysis before determining the frequency of Th17 cells; this can be accomplished by determining intracellular levels of one or more markers of apoptosis.

Sample

The immune cells may be from any sample that contains immune cells. The sample may be from a human or from an animal; in certain embodiments, the sample is from a human. In certain cases, the human may be an individual suffering from, or suspected of or potentially suffering from, a condition, such as an autoimmune condition or a cancer. In certain embodiments, the sample is from a human suffering from a condition, such as an autoimmune condition or a cancer, who is potentially undergoing a treatment, or is undergoing a treatment. For example, in certain embodiments, the sample is from a cancer patient for whom it is being determined whether or not to administer an immunomodulatory treatment, such as treatment with a checkpoint inhibitor, e.g., ipilimumab, or who is currently undergoing such treatment. The sample can be a blood or blood-derived sample; one convenient type of sample is a peripheral blood mononuclear cell (PBMC) sample. The sample can be a tissue or tissue-derived sample, such as a tumor infiltrating lymphocytes (TILS) sample. Any other suitable sample amenable to activation and analysis may also be used. The sample may be fresh or may have been stored under suitable conditions (e.g., frozen and thawed when used in the assay).

Activation

Immune cells in the sample are contacted with one or more TCR modulators. TCR modulators are well known in the art and include, for example, anti-CD3 antibody, alone or in combination with anti-CD28 antibody. As used herein, an “activator of Th17 differentiation” includes substances which alone or in combination with other substances, when contacted with a population of immune cells that includes T cells, generally will move the population toward a population with a higher proportion of Th17 cells than before the contact. For example, a TCR activator, in combination with cytokines that promote Th17 cells and/or inhibitors of other cell types, would, in this sense, be an activator of Th17 differentiation.

In certain embodiments, immune cells from the sample are also contacted with one or more substances that activate cells, e.g., non-T cells, to produce substances that modulate T cells. Such substances can include one or more TLR activators. TLR activators are also well-known in the art, and include TLR4 activators such as lipopolysaccharide (LPS) and TLR7/8 activators such as R848.

Additional elements may also be used to contact the cells, e.g., to bias the cells toward a Th17 subpopulation and/or away from other Th subpopulations, such as away from a Th1 or Th2 phenotype. These can include, e.g., anti-IL-4 and/or anti-IFNg. Additionally or alternatively, these can include TNFa, IL-1b, IL-6, IL-21, IL-23, and/or TGF-b1.

Incubation

Cells are incubated under any suitable conditions and for any suitable time period. The cells may be incubated at 30 to 42° C., for example, 32-40° C., such as 35-39° C.; typically, cells are incubated at or around 37° C., such as at 37° C. The time period of incubation may be any suitable time period. Surprisingly, the inventors have found that under certain conditions, such as with TLR stimulation, the suitable or optimal time period for determining polarization of cells to the Th17 phenotype can be 6 days or less, for example, 3-6 days, or 4-6 days, or 4.5-5.5 days, or 5 days. However, if TLR stimulation is not used, longer time periods of incubation can be used.

Assays

Cells are typically assayed on a single cell basis. Any suitable method may be used, for example flow cytometry or mass cytometry. In addition, or alternatively, extracellular levels of one or more markers (e.g., IL-17), and/or assays of cell lysates may be used.

Th17 cell frequency can be determined by any suitable method. Commonly, cells are first incubated under conditions that stimulate IL-17 production and also prevent its export into the extracellular medium, for example, by contacting the cells with phorbol 12-myristate 13-acetate (PMA) and ionomycin for about an hour followed by Brefeldin A for an additional four hours. The cells are then fixed, permeabilized, and contacted with detectable binding elements, e.g., labeled antibodies, for the elements of interest. These include IL-17, such as IL-17A, IL-17F, or IL-171F, e.g., IL-17A. Cell surface markers can include markers for T cells and T cell subpopulations, such as CD3, CD4, and CD8. Additional or alternative intracellular markers include IFNg and/or TNFa can be assayed in the single cells.

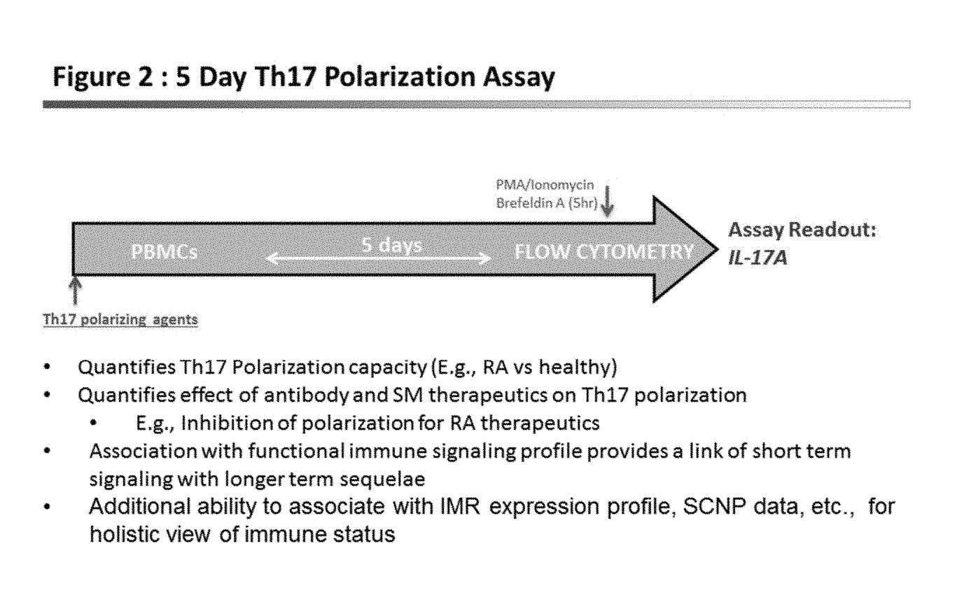

An exemplary Th17 polarization assay is shown in FIG. 2.

Various factors, such as handling, etc., may adversely affect cell health, so that it is advantageous in certain embodiments to exclude unhealthy cells from analysis, e.g., cells undergoing apoptosis. Although these cells may still be alive, they are thought to be no longer in a condition to yield useful information, and the assay can be improved by excluding them. Thus in certain embodiments, one or more intracellular markers of cell health, e.g., one or more markers of apoptosis, may be used to exclude cells from the analysis. Any suitable marker may be used, such as those described in PCT Publication No. WO2012024546. In certain embodiments, c-PARP is used as a marker of apoptosis, and cells with intracellular levels of c-PARP above a certain threshold are excluded from the analysis.

Cells may be gated to determine one or more cell populations. For example, cells may initially be gated by forward scatter (FSC) and side scatter (SSC) to remove debris and define intact cells. Cells may then be gated into, e.g., T cell populations and subpopulations, optionally using one or more markers of cell health to exclude apoptotic cells, such as: T Cells=CD3+ cPARP-; CD4+ T Cells=CD3+ CD4+ cPARP-; CD8+ T Cells=CD3+ CD8+ cPARP-; CD4−CD8− T Cells=CD3+CD4−CD8− cPARP-. Then various populations positive for intracellular cytokines may be defined. (such as IL-17A+ cells within CD4+ T Cells). Here is an example for IL-17A positivity determination, that includes the optional cell health marker c-PARP: CD4+IL-17A+ T Cells=IL-17A+ CD3±CD4-r cPARP-; CD8+IL-17A+I′ Cells=IL-17A CD3+ CD8+ cPARP-; CD4− CD8-IL-17A+ T Cells=IL-17A CD3+ CD4 CD8− cPARP-. A similar process can be repeated with with other intracellular cytokines (e.g., IFN □□□ TNFa.).

The Th17 cell frequency is typically expressed as % CD4 cells expressing IL-17, though any suitable expression may be used. Similar frequencies for other cell types, such as Th1 phenotype, may also be used in addition to, or as an alternative to, the Th17 subtype, in some analyses. The Th17 (or other phenotype) frequency may be determined before stimulation and incubation, after stimulation and incubation, or the before may be combined with the after to give an increase in Th17 phenotype with stimulation.

Using Intracellular Activatable Elements as an Alternative or in Addition to Th17 Polarization

In some cases, intracellular levels of one or more activatable elements, such as a phosphorylatable protein or a protein subject to cleavage, may be used in addition to or as an alternative to stimulation and incubation of cells. Modulation of the cells may be used and the level of the one or more intracellular activatable elements may be determined after modulation and compared to that before. Typically in these cases, the modulation and incubation is on the order of hours or minutes, e.g., 5-120 minutes. For a further description of intracellular activatable elements, modulation, detection, etc., see U.S. Pat. Nos. 8,962,263; 8,865,420; 8,394,599; 8,309,316; 8,227,202; 8,198,037; 9,182,385; 9,034,257. See also FIG. 1 for an overview of exemplary modulators and intracellular activatable elements. In certain embodiments of the invention, cells are contacted with a cytokine, such as IL-4 (Th2 polarizing cytokine), IFNg (Th1 polarizing cytokine), IL-6 (Th-17 polarizing cytokine), and/or IL-23 (Th-17 polarizing cytokines), and the levels of one or more p-STATS determined, such as the level of one or more of p-STAT1, p-STAT3, and/or p-STAT6. In certain embodiments, cells are contacted with a TLR7/8 activator, such as R848, optionally with additional cytokine stimulation, such as IL-1b, and the levels of one or more intracellular activatable elements determined, such as the levels of p-p38, p-ERK, IKBa, p-S6, p-TBK1, and/or p-AKT. In certain embodiments, cells are contacted with a TLR4 activator, such as LPS, and the levels of one or more intracellular activatable elements determined, such as the levels of, p-ERK, p-TBK1, and/or p-AKT.

Diagnosis, Prognosis, Prediction, Monitoring.

In one aspect, the invention provides methods and compositions directed at diagnosing, prognosing, predicting, and/or monitoring a condition. The condition may be any suitable condition of interest, for example, a pathological condition such as an autoimmune condition or a cancer.

Examples of autoimmune conditions or disorders include, but are not limited to: arthritis, including rheumatoid arthritis, acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, juvenile-onset rheumatoid arthritis, osteoarthritis, arthritis chronica progrediente, arthritis deformans, polyarthritis chronica primaria, reactive arthritis, and ankylosing spondylitis; inflammatory hyperproliferative skin diseases; psoriasis, such as plaque psoriasis, gutatte psoriasis, pustular psoriasis, and psoriasis of the nails; atopy, including atopic diseases such as hay fever and Job's syndrome; dermatitis, including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis; x-linked hyper IgM syndrome; allergic intraocular inflammatory diseases; urticaria, such as chronic allergic urticaria, chronic idiopathic urticaria, and chronic autoimmune urticaria; myositis; polymyositis/dermatomyositis; juvenile dermatomyositis; toxic epidermal necrolysis; scleroderma, including systemic scleroderma; sclerosis, such as systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis; neuromyelitis optica (NMO); inflammatory bowel disease (IBD), including Crohn's disease, autoimmune-mediated gastrointestinal diseases, colitis, ulcerative colitis, colitis ulcerosa, microscopic colitis, collagenous colitis, colitis polyposa, necrotizing enterocolitis, transmural colitis, and autoimmune inflammatory bowel disease; bowel inflammation; pyoderma gangrenosum; erythema nodosum; primary sclerosing cholangitis; respiratory distress syndrome, including adult or acute respiratory distress syndrome (ARDS); meningitis; inflammation of all or part of the uvea; iritis; choroiditis; an autoimmune hematological disorder; rheumatoid spondylitis; rheumatoid synovitis; hereditary angioedema; cranial nerve damage, as in meningitis; herpes gestationis; pemphigoid gestationis; pruritis scroti; autoimmune premature ovarian failure; sudden hearing loss due to an autoimmune condition; IgE-mediated diseases, such as anaphylaxis and allergic and atopic rhinitis; encephalitis, such as Rasmussen's encephalitis and limbic and/or brainstem encephalitis; uveitis, such as anterior uveitis, acute anterior uveitis, granulomatous uveitis, nongranulomatous uveitis, phacoantigenic uveitis, posterior uveitis, or autoimmune uveitis; glomerulonephritis (GN) with and without nephrotic syndrome, such as chronic or acute glomerulonephritis, primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN; proliferative nephritis; autoimmune polyglandular endocrine failure; balanitis, including balanitis circumscripta plasmacellularis; balanoposthitis; erythema annulare centrifugum; erythema dyschromicum perstans; eythema multiform; granuloma annulare; lichen nitidus; lichen sclerosus et atrophicus; lichen simplex chronicus; lichen spinulosus; lichen planus; lamellar ichthyosis; epidermolytic hyperkeratosis; premalignant keratosis; pyoderma gangrenosum; allergic conditions and responses; allergic reaction; eczema, including allergic or atopic eczema, asteatotic eczema, dyshidrotic eczema, and vesicular palmoplantar eczema; asthma, such as asthma bronchiale, bronchial asthma, and auto-immune asthma; conditions involving infiltration of T cells and chronic inflammatory responses; immune reactions against foreign antigens such as fetal A-B-O blood groups during pregnancy; chronic pulmonary inflammatory disease; autoimmune myocarditis; leukocyte adhesion deficiency; lupus, including lupus nephritis, lupus cerebritis, pediatric lupus, non-renal lupus, extra-renal lupus, discoid lupus and discoid lupus erythematosus, alopecia lupus, systemic lupus erythematosus (SLE), cutaneous SLE, subacute cutaneous SLE, neonatal lupus syndrome (NLE), and lupus erythematosus disseminatus; juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, and diabetic large-artery disorder; immune responses associated with acute and delayed hypersensitivity mediated by cytokines and T-lymphocytes; tuberculosis; sarcoidosis; granulomatosis, including lymphomatoid granulomatosis; Wegener's granulomatosis; agranulocytosis; vasculitides, including vasculitis, large-vessel vasculitis, polymyalgia rheumatica and giant-cell (Takayasu's) arteritis, medium-vessel vasculitis, Kawasaki's disease, polyarteritis nodosa/periarteritis nodosa, microscopic polyarteritis, immunovasculitis, CNS vasculitis, cutaneous vasculitis, hypersensitivity vasculitis, necrotizing vasculitis, systemic necrotizing vasculitis, ANCA-associated vasculitis, Churg-Strauss vasculitis or syndrome (CSS), and ANCA-associated small-vessel vasculitis; temporal arteritis; aplastic anemia; autoimmune aplastic anemia; Coombs positive anemia; Diamond Blackfan anemia; hemolytic anemia or immune hemolytic anemia, including autoimmune hemolytic anemia (AIHA), pernicious anemia (anemia perniciosa); Addison's disease; pure red cell anemia or aplasia (PRCA); Factor VIII deficiency; hemophilia A; autoimmune neutropenia; pancytopenia; leukopenia; diseases involving leukocyte diapedesis; CNS inflammatory disorders; multiple organ injury syndrome, such as those secondary to septicemia, trauma or hemorrhage; antigen-antibody complex-mediated diseases; anti-glomerular basement membrane disease; anti-phospholipid antibody syndrome; allergic neuritis; Behcet's disease/syndrome; Castleman's syndrome; Goodpasture's syndrome; Reynaud's syndrome; Sjogren's syndrome; Stevens-Johnson syndrome; pemphigoid, such as pemphigoid bullous and skin pemphigoid, pemphigus, pemphigus vulgaris, pemphigus foliaceus, pemphigus mucus-membrane pemphigoid, and pemphigus erythematosus; autoimmune polyendocrinopathies; Reiter's disease or syndrome; thermal injury; preeclampsia; an immune complex disorder, such as immune complex nephritis, and antibody-mediated nephritis; polyneuropathies; chronic neuropathy, such as IgM polyneuropathies and IgM-mediated neuropathy; thrombocytopenia (as developed by myocardial infarction patients, for example), including thrombotic thrombocytopenic purpura (TTP), post-transfusion purpura (PTP), heparin-induced thrombocytopenia, autoimmune or immune-mediated thrombocytopenia, idiopathic thrombocytopenic purpura (ITP), and chronic or acute ITP; scleritis, such as idiopathic cerato-scleritis, and episcleritis; autoimmune disease of the testis and ovary including, autoimmune orchitis and oophoritis; primary hypothyroidism; hypoparathyroidism; autoimmune endocrine diseases, including thyroiditis, autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes, autoimmune polyglandular syndromes, and polyglandular endocrinopathy syndromes; paraneoplastic syndromes, including neurologic paraneoplastic syndromes; Lambert-Eaton myasthenic syndrome or Eaton-Lambert syndrome; stiff-man or stiff-person syndrome; encephalomyelitis, such as allergic encephalomyelitis, encephalomyelitis allergica, and experimental allergic encephalomyelitis (EAE); myasthenia gravis, such as thymoma-associated myasthenia gravis; cerebellar degeneration; neuromyotonia; opsoclonus or opsoclonus myoclonus syndrome (OMS); sensory neuropathy; multifocal motor neuropathy; Sheehan's syndrome; hepatitis, including autoimmune hepatitis, chronic hepatitis, lupoid hepatitis, giant-cell hepatitis, chronic active hepatitis, and autoimmune chronic active hepatitis; lymphoid interstitial pneumonitis (LIP); bronchiolitis obliterans (non-transplant) vs NSIP; Guillain-Barre syndrome; Berger's disease (IgA nephropathy); idiopathic IgA nephropathy; linear IgA dermatosis; acute febrile neutrophilic dermatosis; subcorneal pustular dermatosis; transient acantholytic dermatosis; cirrhosis, such as primary biliary cirrhosis and pneumonocirrhosis; autoimmune enteropathy syndrome; Celiac or Coeliac disease; celiac sprue (gluten enteropathy); refractory sprue; idiopathic sprue; cryoglobulinemia; amylotrophic lateral sclerosis (ALS; Lou Gehrig's disease); coronary artery disease; autoimmune ear disease, such as autoimmune inner ear disease (AIED); autoimmune hearing loss; polychondritis, such as refractory or relapsed or relapsing polychondritis; pulmonary alveolar proteinosis; Cogan's syndrome/nonsyphilitic interstitial keratitis; Bell's palsy; Sweet's disease/syndrome; rosacea autoimmune; zoster-associated pain; amyloidosis; a non-cancerous lymphocytosis; a primary lymphocytosis, including monoclonal B cell lymphocytosis (e.g., benign monoclonal gammopathy and monoclonal gammopathy of undetermined significance, MGUS); peripheral neuropathy; channelopathies, such as epilepsy, migraine, arrhythmia, muscular disorders, deafness, blindness, periodic paralysis, and channelopathies of the CNS; autism; inflammatory myopathy; focal or segmental or focal segmental glomerulosclerosis (FSGS); endocrine opthalmopathy; uveoretinitis; chorioretinitis; autoimmune hepatological disorder; fibromyalgia; multiple endocrine failure; Schmidt's syndrome; adrenalitis; gastric atrophy; presenile dementia; demyelinating diseases, such as autoimmune demyelinating diseases and chronic inflammatory demyelinating polyneuropathy; Dressler's syndrome; alopecia areata; alopecia totalis; CREST syndrome (calcinosis, Raynaud's phenomenon, esophageal dysmotility, sclerodactyly, and telangiectasia); male and female autoimmune infertility (e.g., due to anti-spermatozoan antibodies); mixed connective tissue disease; Chagas' disease; rheumatic fever; recurrent abortion; farmer's lung; erythema multiforme; post-cardiotomy syndrome; Cushing's syndrome; bird-fancier's lung; allergic granulomatous angiitis; benign lymphocytic angiitis; Alport's syndrome; alveolitis, such as allergic alveolitis and fibrosing alveolitis; interstitial lung disease; transfusion reaction; leprosy; malaria; Samter's syndrome; Caplan's syndrome; endocarditis; endomyocardial fibrosis; diffuse interstitial pulmonary fibrosis; interstitial lung fibrosis; pulmonary fibrosis; idiopathic pulmonary fibrosis; cystic fibrosis; endophthalmitis; erythema elevatum et diutinum; erythroblastosis fetalis; eosinophilic faciitis; Shulman's syndrome; Felty's syndrome; flariasis; cyclitis, such as chronic cyclitis, heterochronic cyclitis, iridocyclitis (acute or chronic), or Fuch's cyclitis; Henoch-Schonlein purpura; sepsis; endotoxemia; pancreatitis; thyroxicosis; Evan's syndrome; autoimmune gonadal failure; Sydenham's chorea; post-streptococcal nephritis; thromboangitis ubiterans; thyrotoxicosis; tabes dorsalis; chorioiditis; giant-cell polymyalgia; chronic hypersensitivity pneumonitis; keratoconjunctivitis sicca; epidemic keratoconjunctivitis; idiopathic nephritic syndrome; minimal change nephropathy; benign familial and ischemia-reperfusion injury; transplant organ reperfusion; retinal autoimmunity; joint inflammation; bronchitis; chronic obstructive airway/pulmonary disease; silicosis; aphthae; aphthous stomatitis; arteriosclerotic disorders; aspermiogenese; autoimmune hemolysis; Boeck's disease; cryoglobulinemia; Dupuytren's contracture; endophthalmia phacoanaphylactica; enteritis allergica; erythema nodosum leprosum; idiopathic facial paralysis; febris rheumatica; Hamman-Rich's disease; sensoneural hearing loss; haemoglobinuria paroxysmatica; hypogonadism; ileitis regionalis; leucopenia; mononucleosis infectiosa; traverse myelitis; primary idiopathic myxedema; nephrosis; ophthalmia symphatica; orchitis granulomatosa; pancreatitis; polyradiculitis acuta; pyoderma gangrenosum; Quervain's thyreoiditis; acquired spenic atrophy; non-malignant thymoma; vitiligo; toxic-shock syndrome; food poisoning; conditions involving infiltration of T cells; leukocyte-adhesion deficiency; immune responses associated with acute and delayed hypersensitivity mediated by cytokines and T-lymphocytes; diseases involving leukocyte diapedesis; multiple organ injury syndrome; antigen-antibody complex-mediated diseases; antiglomerular basement membrane disease; allergic neuritis; autoimmune polyendocrinopathies; oophoritis; primary myxedema; autoimmune atrophic gastritis; sympathetic ophthalmia; rheumatic diseases; mixed connective tissue disease; nephrotic syndrome; insulitis; polyendocrine failure; autoimmune polyglandular syndrome type I; adult-onset idiopathic hypoparathyroidism (AOIH); cardiomyopathy such as dilated cardiomyopathy; epidermolisis bullosa acquisita (EBA); hemochromatosis; myocarditis; nephrotic syndrome; primary sclerosing cholangitis; purulent or nonpurulent sinusitis; acute or chronic sinusitis; ethmoid, frontal, maxillary, or sphenoid sinusitis; an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils; anaphylaxis; seronegative spondyloarthritides; polyendocrine autoimmune disease; sclerosing cholangitis; chronic mucocutaneous candidiasis; Bruton's syndrome; transient hypogammaglobulinemia of infancy; Wiskott-Aldrich syndrome; ataxia telangiectasia syndrome; angiectasis; autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization; allergic hypersensitivity disorders; glomerulonephritides; reperfusion injury; ischemic re-perfusion disorder; reperfusion injury of myocardial or other tissues; lymphomatous tracheobronchitis; inflammatory dermatoses; dermatoses with acute inflammatory components; multiple organ failure; bullous diseases; renal cortical necrosis; acute purulent meningitis or other central nervous system inflammatory disorders; ocular and orbital inflammatory disorders; granulocyte transfusion-associated syndromes; cytokine-induced toxicity; narcolepsy; acute serious inflammation; chronic intractable inflammation; pyelitis; endarterial hyperplasia; peptic ulcer; valvulitis; and endometriosis. In certain embodiments, the condition is rheumatoid arthritis (RA).

In certain embodiments, the condition is cancer. The cancer may produce solid tumors or hematological tumors. Cancers that produce solid tumors include adrenal cortical cancer, anal cancer, bile duct cancer (e.g. peripheral cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating, lobular carcinoma, lobular carcinoma in, situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adnocarcinoma, clear cell), esophagus cancer, gallbladder cancer (mucinous adenocarcinoma, small cell carcinoma), gastrointestinal carcinoid tumors (e.g. choriocarcinoma, chorioadenoma destruens), Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma). Primary cancers and metastases as well as cancers of unknown primary are included.

Cancers that produce hematological tumors include but are not limited to Non-Hodgkin Lymphoma, Hodgkin or other lymphomas, acute or chronic leukemias, and multiple myeloma. In certain embodiments, the cancer is non-B lineage derived, such as Acute myeloid leukemia (AML), Chronic Myeloid Leukemia (CML), non-B cell Acute lymphocytic leukemia (ALL), or non-B cell lymphomas. In certain embodiments, the cancer is a B-Cell or B cell lineage derived cancer. Examples of B-Cell or B cell lineage cancers include but are not limited to Chronic Lymphocytic Leukemia (CLL), B lymphocyte lineage leukemia, B lymphocyte lineage lymphoma, and Multiple Myeloma. Other conditions within the scope of the present invention include, but are not limited to, cancers such as gliomas, lung cancer, colon cancer and prostate cancer. In certain embodiments, the cancer is melanoma, small cell lung cancer, non-small cell lung carcinoma, bladder cancer, or prostate cancer. In certain embodiments, the condition is melanoma.

In certain embodiments, the methods include inducing polarization of a T cell population, such as Th17 polarization, in immune cells from a sample from an individual, as described herein, and from the results of the polarization, e.g., change in frequency of Th17 cells, diagnosing, prognosing, predicting (e.g., predicting response to treatment), and/or monitoring (e.g., monitoring a condition or monitoring a treatment) the condition. For example, immune cells from a sample, e.g., a PBMC sample, can be treated with a TCR activator, and/or other agents as described herein, such as one or more TLR activators, one or more cytokines, one or more cytokine inhibitors, and incubated for a period of time (such as less than 6 days, e.g., if a TLR activator is used), then the frequency of a T cell population, or the change in frequency of a T cell population, e.g., a Th17 population, can be determined, based on determinations of one or more intracellular markers in single cells. The frequency or change in frequency of the T cell population can be used, alone or in combination with other factors, to diagnose, prognose, predict, or monitor the condition, such as an autoimmune condition, or cancer.

In certain embodiments, the invention provides a method of predicting whether or not an individual will develop an adverse effect as a result of treatment with an immunomodulatory agent by determining the frequency of Th17 cells in a sample from the individual. Any method as described herein may be used to determine the frequency of Th17 cells, for example, activating immune cells in the sample and incubating for a period of time, then determining the increase in frequency of Th17 cells by determining levels of IL-17A and cell surface marker in individual cells; alternatively or additionally, the frequency of Th17 cells in the sample before activation may also be used. Activation may be as described herein, e.g., with treatment including TCR activators, optionally including one or more TLR activators. The period of incubation can be any period described herein, e.g., less than 6 days. Cells can be gated to remove unhealthy cells, e.g., cells undergoing apoptosis, as described herein. The immunomodulatory agent can be a checkpoint inhibitor, such as ipilimumab, mivolumab, or atezolizumab, e.g, ipilimumab; e.g., the individual can be an individual suffering from a cancer, such as melanoma, small cell lung cancer, non-small cell lung carcinoma, bladder cancer, or prostate cancer, who is being considered for treatment with ipilimumab and/or who is undergoing treatment with ipilimumab. The adverse effect can be a gastrointestinal effect, e.g., colitis. The method can be used before and/or during treatment. In some cases, the results of the test (e.g., frequency of Th17 cells above or below a certain threshold, or within a certain range, or IL-17, i.e., intracellular IL-17 levels, such as intracellular levels measured on a single cell basis, levels above or below a certain threshold or in a certain range) are used to determine not to treat the individual with ipilimumab, or to modify upcoming treatment with ipilimumab, such as to not treat, use a different, e.g., lower, dose, use a different frequency of dosage, additional agents (e.g., anti-IL-17 agents, and/or agents that ameliorate colitis such as anti-inflammatory agents such as steroid agents, including anti-inflammatory agents such as steroid agents acting specifically on the GI tract such as non-absorbable anti-inflammatory agents), alternative agents, or the like. In an individual being treated with ipilimumab, treatment can be monitored periodically, at any suitable interval, e.g., monthly, and Th17 cell frequency (and/or IL-17 levels) can be monitored as an absolute level, and additionally or alternatively, compared to baseline levels, e.g., by the methods described herein; increases or decreases in absolute levels, and/or above or below baseline levels greater than a certain amount and/or at greater than a certain rate, can signal the likely onset of colitis, and treatment modulated, if necessary. See Example 1 for correlations of Th17 frequencies with colitis in ipilimumab-treated patients. This can include halting treatment, adjusting dosage, adjusting timing of dosage, including additional agents, and the like.

In some cases, rather than determining frequency of Th-17 cells, e.g., by determining cells above a certain threshold of IL-17A, intracellular levels of IL-17, such as IL-17A, may be determined regardless of threshold and the decision to treat or not treat, or to modulate treatment, may be based on this determination. In some cases, in addition to, or as an alternative to, determining Th17 cell frequency, the levels of one or more activatable elements in single cells, optionally in response to treatment with a modulator, may be determined and used to predict whether or not an individual will develop an adverse effect as a result of treatment with an immunomodulatory agent; activatable elements and modulators may be any such as described herein. Extracellular levels of IL-17, e.g., levels of IL-17 secreted into the medium, may also be used in the determination. In addition, serum levels of IL-17 or other markers may be taken into consideration. Other relevant clinical factors may be considered, such as age, gender, race, previous treatment, family history, genetic factors, previous or concurrent gastrointestinal disorders, presence of other conditions, and the like.

In these and other methods and compositions described herein, additional factors may be used in combination with the T cell population assays, e.g., Th17 cell assays. These include regulatory T cell (Treg) numbers, Th17:Treg ratio, serum IL-10 levels, serum IL-17 levels, and/or SCNP signaling, such as cytokine→STAT signaling, such as cytokine→pSTAT3 signaling.

Screening Agents

The invention also provides methods and compositions for screening agents, for example, for screening agents that are potentially useful as drugs to treat certain conditions, such as autoimmune conditions or cancer. Such conditions have been described elsewhere herein. The methods and compositions can be used to determine potential beneficial effects and/or potential adverse effects.

In certain embodiments, the invention provides a method for screening agents that includes contacting immune cells with an activator of Th17 differentiation, contacting the cells with an agent, incubating the cells for a period of time, and determining the frequency of Th17 cells after incubation, with and without the agent. An agent that inhibits Th17 polarization is a potential candidate for use as a treatment in condition in which the Th17, inflammatory, phenotype is present. On the other hand, an agent that is otherwise potentially useful as a treatment for a condition may demonstrate a high level of Th17 polarization, indicating the potential for adverse effects involving this T cell population.

In addition, the methods may be used to monitor effects of drugs during treatment, for example, in clinical trials, in which individuals suffering from a condition, e.g., an autoimmune condition such as rheumatoid arthritis or a cancer such as melanoma, non-small cell lung carcinoma, small cell lung cancer, bladder cancer, or prostate cancer, are treated with a drug. A Th17 polarization assay, as described herein, can be conducted before the trial. Such results can be used as a baseline and/or, e.g., to stratify patients and potentially to determine which patients should be given the drug. A Th17 polarization assay, as described herein, can also be conducted at intervals and/or at the end of the trial, to track the effect of the drug over the course of treatment, which can include beneficial effects (e.g., reduction in tendency of cells to polarize to the Th17 phenotype) and/or adverse effects (e.g., increase in tendency of cells to polarize to the Th17 phenotype).

In addition, the invention provides methods and compositions (such as kits, see below), that can be used with an agent intended to treat a condition to determine likelihood of success of treatment with the agent and/or likelihood of adverse effects with the agent, e.g., likelihood of an adverse effect that is related to the Th17 phenotype, such as colitis.

Results of Th17 and/or Th1 polarization assays can also be used with patients receiving a particular agent to determine whether or not a combination therapy is warranted. For example, RA patients treated with TNF inhibitors exhibited greater polarization toward the Th17 phenotype after 5 days of incubation with various combinations, especially with incubation with TCR and TLR activators (see Example 2). In such patients, combination therapy with an anti-IL-17 agent may be warranted to prevent adverse effects from the greater tendency toward the Th17 phenotype.

Exemplary agent types that can be useful in conditions in which the Th17 cell phenotype plays a role include anti-IL-23 agents, anti-IL-17 agents, PI3 kinase inhibitors, anti-TNFa, and agents with a dual effect, e.g., anti-TNFa/IL-17.

Kits

In certain embodiments, the invention provides kits.

For example in certain embodiments, the invention provides a kit comprising (i) an activator of T cells; (ii) a modulator of a non-T cell population; (iii) a detectable binding element, such as an antibody, specific for IL-17; (iv) an agent for inducing IL-17 formation. The modulator of a non-T cell population can comprise an activator of a non-T cell population, for example, a toll-like receptor (TLR) activator, such as a TLR4 activator, e.g., LPS, and/or a TLR7/8 activator, e.g., R848. The kit can further comprise one or more detectable binding elements to a cell surface marker, for example, a cell surface marker selected from the group consisting of CD3, CD4, CD8, and combinations thereof. In certain embodiments, the activator of T cells is a TCR activator, such as anti-CD3, anti-CD28, or a combination thereof. The kit can further comprise a detectable binding element for a marker of cell health, such as a marker of apoptosis, e.g., cPARP. The kit can further comprise instructions. The kit can further comprise packaging to hold components of the kit.

In certain embodiments the invention provides a kit comprising (i) an activator of T cells; (ii) a detectable binding element for a marker of apoptosis, for example, c-PARP; (iii) a detectable binding element specific for IL-17; (iv) an agent for inducing IL-17 formation. The kit can further comprise a modulator of a non-T cell population, The modulator of a non-T cell population can comprise an activator of a non-T cell population, for example, a toll-like receptor (TLR) activator, such as a TLR4 activator, e.g., LPS, and/or a TLR7/8 activator, e.g., R848. The kit can further comprise one or more detectable binding elements to a cell surface marker, for example, a cell surface marker selected from the group consisting of CD3, CD4, CD8, and combinations thereof. In certain embodiments, the activator of T cells is a TCR activator, such as anti-CD3, anti-CD28, or a combination thereof. The kit can further comprise instructions. The kit can further comprise packaging to hold components of the kit.

While in many cases detectable binding elements are described as labeled with fluorophores (either directly, e.g., through attachment to a primary antibody, or indirectly, e.g., through attachment to a secondary antibody), it will be understood that the description is equally applicable to detectable binding elements labeled with mass tags, for detection by mass cytometry.

The binding elements of kits of the invention can be conjugated to a solid support. In some embodiments, binding elements are immobilized using beads analogous to those known and used for standardization in flow cytometry. Attachment of a multiplicity of binding elements to beads may be done by methods known in the art. Such conjugated beads may be contacted with sample, preferably cell extract, under conditions that allow for a multiplicity analytes, if present, to bind to the multiplicity of immobilized binding elements. Calibration beads may be added to the kits for calibration and performance monitoring of a fluorescence detector. Detailed discussion of the usage of calibration beads disclosed in U.S. Ser. No. 61/176,420 is hereby incorporated by reference in its entity.

Kits of the present invention can also include one or more reagents or supplies that are useful in the invention, such as fixatives, permeabilizing agent, buffers, containers, plates, instructions, and the like.

In certain embodiments, kits of the present invention also comprise fixatives to preserve or “freeze” a cell in a certain state, preferably so that an accurate representation of the structure of the cell is maintained. Cells may be fixed by any of a variety of suitable chemical and physical methods. The commonly used cell fixatives include, but not limited to formaldehyde, paraformaldehyde, glutaraldehyde, acetic acid, picric acid, methanol, ethanol, and acetone. Preferred fixatives comprised 0.756%-0.85% formaldehyde, 25.4-30 mM DNBS, 6.9-6.92% DMSO and 0.086-0.095% TWEEN™ 20 detergent, although many variations are described.

In certain embodiments, kits can comprise wash buffers containing fixatives to fix a cell after stimulation with a modulator. Wash buffers are well known in the art. Prior art examples disclosed in U.S. Pat. No. 7,326,577 and U.S. Pub. No. 2006/0141549 are hereby incorporated by reference in their entireties. One exemplary fixation buffer suitable for whole blood samples is BD™ Phosflow Lyse/Fix Buffer (BD Biosciences, Franklin Lakes, N.J.).

Fixatives have been used for detection of both surface and intracellular antigens. See, Francis C. & Connelly M. C., Rapid single-step method for flow cytometric detection of surface and intracellular antigens using whole blood, Cytometry (1996) 25(1):58-70. Current fixatives revolve primarily around alcohol and formaldehyde/paraformaldehyde, Jacobberger, J W, Flow Cytometric Analysis of Intracellular Protein Epitopes. Immunophenotyping (2000) 361-409. The fixative described by Connelly (Pizzolo, G, et al. Detection of membrane and intracellular antigens by flow cytometry following ORTHO PermeaFix fixation. Leukemia. (1994) 8(4):672-76) is the best single step fixative and permeation agent discovered to date (see Metso, T, et al., Identification of intracellular markers in induced sputum and bronchoalveolar lavage samples in patients with respiratory disorders and healthy persons. Respir Med. (2002) 6(11):918-26) stating that “Best results were obtained using a commercial reagent Ortho PermeaFix (OPF) for flow cytometry”). It is called Ortho PERMEAFIX™, although that product has been replaced with a new product called PERMIFLOW™ (INVIRION, INC.™ MI). OPF and its variants are well described in U.S. Pat. No. 5,422,277 and U.S. Pat. No. 5,597,688. Preferred fixatives comprised 0.756%-0.85% formaldehyde, 25.4-30 mM DNBS, 6.9-6.92% DMSO and 0.086-0.095% TWEEN™ 20 detergent, although many variations are described.

In certain embodiment, kits of the present invention can further comprise a permeabilizing agent. Permeabilization is performed to facilitate access to cellular cytoplasm or intracellular molecules, components or structures of a cell. In particular, permeabilization can allow a binding element (such as a phospho-specific antibody) to enter into a cell and reach an intracellular concentration much greater than the concentration in the absence of such permeabilizing treatment.

Permeabilization of the cells can be performed by any suitable method (see, for example, C. A. Goncalves et al., Neurochem. Res. (2000) 25:885-894). These methods include, but are not limited to, exposure to a detergent (such as CHAPS, cholic acid, deoxycholic acid, digitonin, n-dodecyl-.beta.-D-maltoside, lauryl sulfate, glycodeoxycholic acid, n-lauroylsarcosine, saponin, and triton X-100) or to an organic alcohol (such as methanol and ethanol). Other permeabilizing methods comprise the use of certain peptides or toxins that render membranes permeable (see, for example, O. Aguilera et al., FEBS Lett. (1999) 462:273-277; and Bussing A. et al., Cytometry (1999) 37:133-139). Permeabilization may also be performed by addition of an organic alcohol to the cells. Selection of an appropriate permeabilizing agent and optimization of the incubation conditions and time can easily be performed by one of ordinary skill in the art. Cells can be permeabilized in the presence of 90% methanol and incubated on ice for 30 minutes. Following this treatment, the assay plate may be stored at −20.degree. C. for up to one month before being analyzed. Permeabilization can occur concurrently with the fixation step. With for example, BD™ Cytofix/Cytoperm (BD Biosciences, Franklin Lakes, N.J.).

In certain embodiments, some of the components of the kits can be lyophilized or frozen in the multi-well plates as part of the kit. The choice of fluorochrome conjugated binding elements for surface markers and intracellular proteins can be designed for one channel or more than one channel to allow the user some flexibility to add their own stain and to allow some customization of the experiment. Kits may also be designed for specific flow cytometer, for example, one for many channels (LSR II-Becton Dickinson), or one for a small number of channels (FACS Canto II-Becton Dickinson).

The kit can further include, where necessary, agents for reducing background interference in a test, control reagents, apparatus for conducting a test, and the like. The kit can be packaged in any suitable manner, typically with all elements in a single container along with a sheet of printed instructions for carrying out the test.

Some embodiments of the invention can additionally comprise software on a CD, a removable hard disk drive, USB or flash drive, or instructions to go to a particular website, implemented with methods for collection, storage, display and querying information on the relationship between modulators, activated elements, and/or cell type, and may further include further correlations on signaling, e.g. signaling data generated by flow cytometry analysis, such as signaling pathways or signaling levels. Some embodiments of the software comprise a graphical user interface (GUI) for displaying, querying and/or filtering the obtained information.

Such kits can also include information, such as protocols, scientific literature references, package insert materials, clinical trial results, and/or summaries of these and the like, which indicate or establish the activities and/or advantages of the composition, and/or which describe optimal concentration, dosing, administration, side effects, drug interactions, or other information useful to the health care provider. Such information can be based on the results of various studies, for example, studies using experimental animals involving in vivo models and studies based on human clinical trials.

In some embodiments, a kit of the present invention can additionally comprise controls and assay preparation protocols

### EXAMPLES

### Example 1

Peripheral blood mononuclear cell samples (PBMC) were obtained from patients suffering from melanoma, before treatment with ipilimumab, and after 6 weeks of treatment with ipilimumab. The cells were treated as described herein to polarize Th17 cells. The frequency of Th17 cells, expressed as % IL17+ CD4+ T cells, was determined. In addition, patients were followed for development of colitis, and for those who developed colitis, the colitis was graded as severity 1, 2, or 3 (no colitis was severity grade 0).

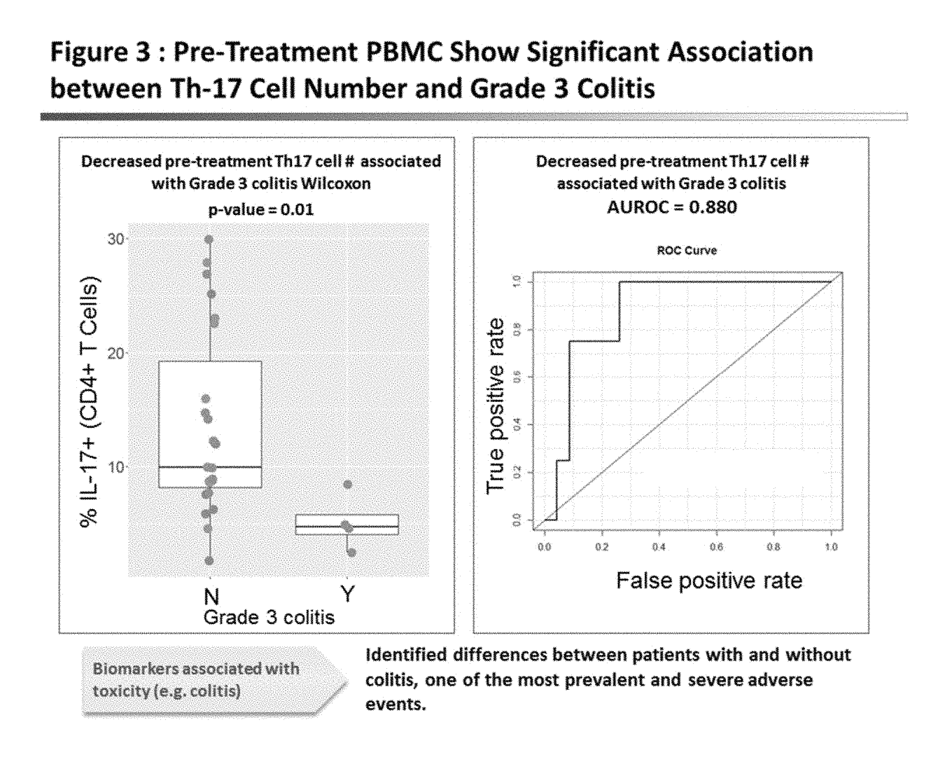

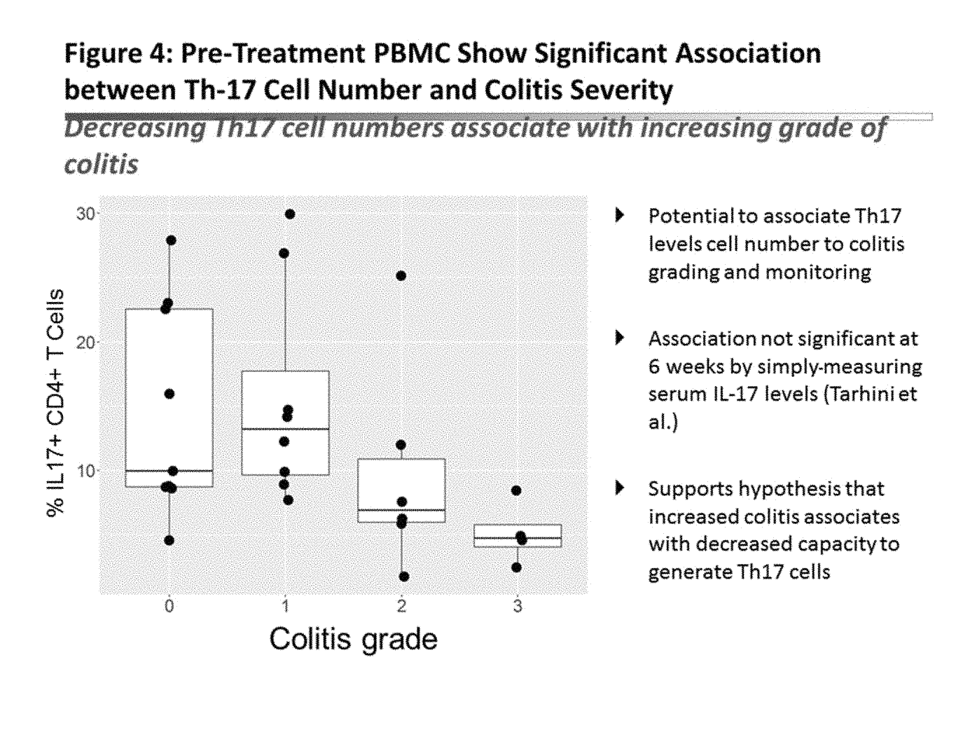

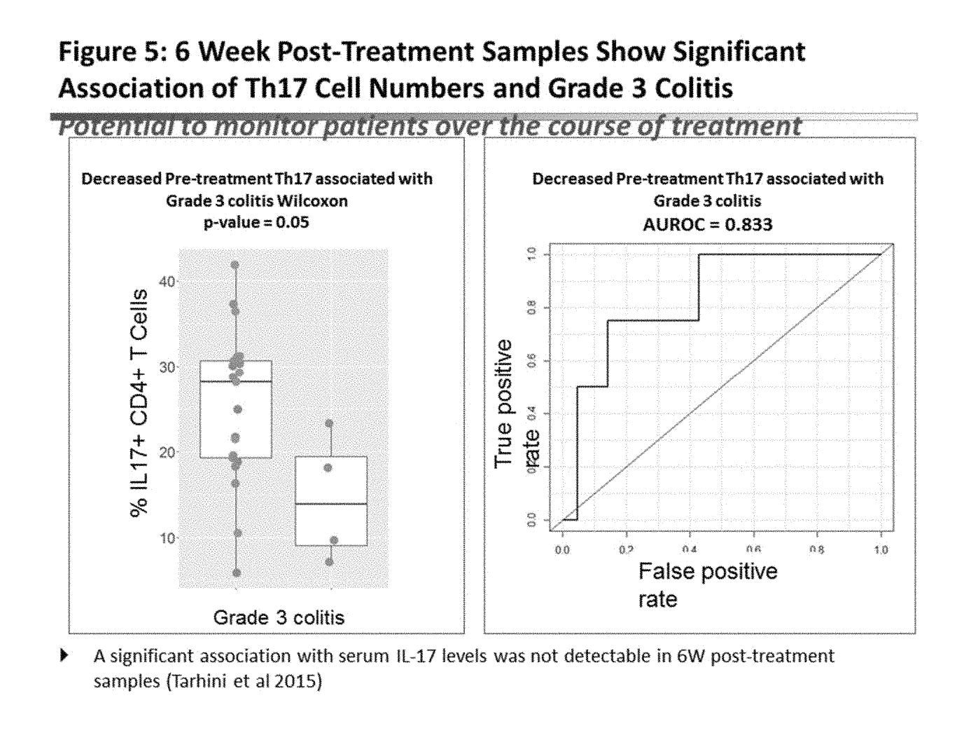

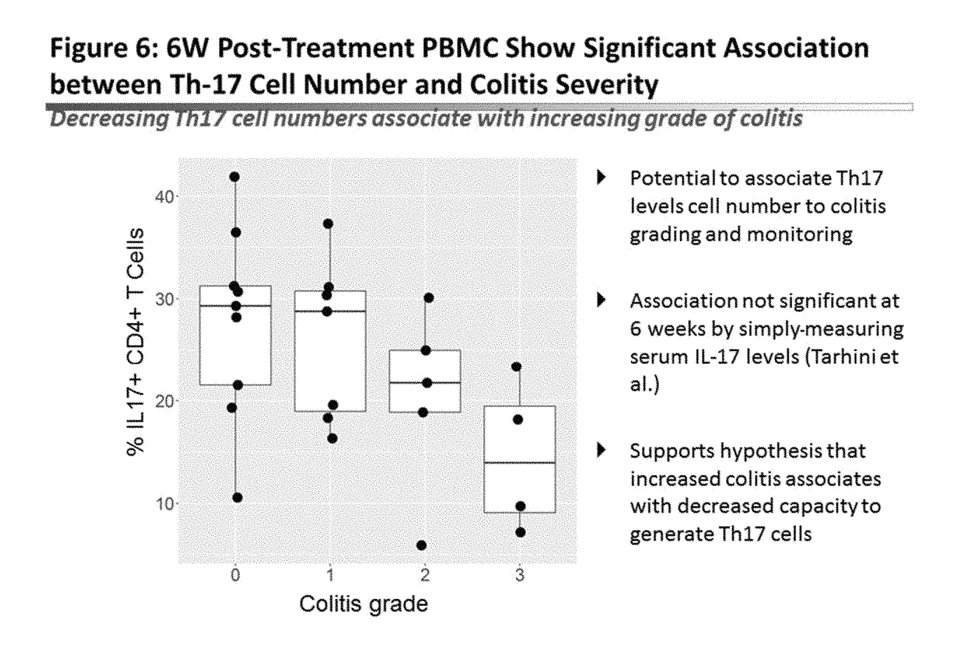

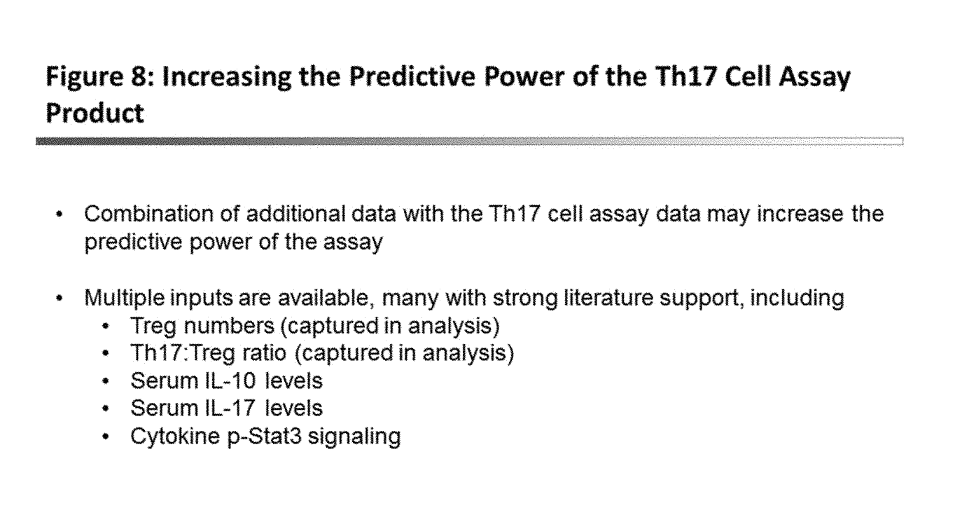

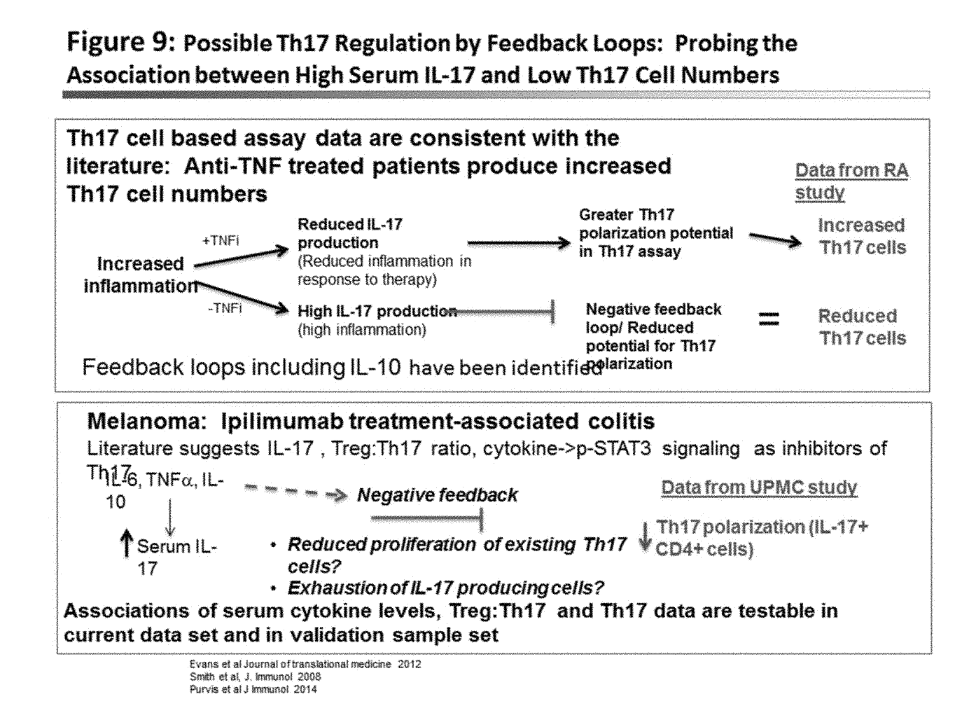

The results are shown in FIGS. 3-6. Surprisingly, for both pre-treatment and 6-week treatment samples, lower Th17 cell frequency was significantly associated with development of Grade 3 colitis; in addition, the magnitude of the decrease in Th17 cell frequency was also associated with the grade of colitis in patients that developed colitis. Previous work has shown that increases in serum IL-17 (which is secreted by Th17 cells) is associated with development of Grade 3 colitis in ipilimumab-treated patients; the magnitude of the increase could not predict Grade of colitis. Thus, it was surprising that the present Example, though it shows a strong association of Th17 cell frequency with colitis development and grade of colitis, the association is negative, not positive. Without being bound by theory, it is thought that the mechanism for this may be as shown in FIG. 9. Additional characteristics which can, in certain embodiments, be used in addition to Th17 frequency to predict adverse effects are shown in FIG. 8. However, regardless of mechanism, these results demonstrate that the methods and compositions of the present invention can be used to predict and/or monitor adverse events in immunotherapy, such as cancer immunotherapy with a checkpoint modulator; e.g., ipilimumab. Clinicians can use such methods and compositions to, e.g., determine whether or not a patient should receive treatment (often in combination with other measures or characteristics), treatment should be modified, or the like, as described elsewhere herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method comprising
(i) contacting immune cells from a sample with one or more activators of T cells;
(ii) incubating the cells for less than 6 days
(iii) determining the frequency of Th17 cells in the sample after the incubation.

2. The method of claim 1 wherein the cells are incubated between 4 and 5.8 days.

3. The method of claim 2 wherein the cells are incubated between 4.5 and 5.5 days.

4. The method of claim 3 wherein the cells are incubated for 5 days.

5. The method of claim 1 wherein the frequency of another Tcell subpopulation is determined.

6. The method of claim 5 wherein the other Tcell subpopulation is a Th1 subpopulation

7. The method of claim 1 further comprising treating the cells with a modulator of a non-T cell population.

8. The method of claim 7 wherein the modulator is an activator of a non-T cell population.

9. The method of claim 8 wherein the activator comprises a toll-like receptor (TLR) activator.

10. The method of claim 9 wherein the TLR activator comprises an activator of TLR4 or an activator of TLR7/8.

11. The method of claim 10 wherein the TLR activator comprises an activator of TLR4 comprising LPS.

12. The method of claim 10 wherein the TLR activator comprises an activator of TLR7/8 comprising R848.

13. The method of claim 1 further comprising determining cell health before determining frequency of Th17 cells, and eliminating cells that are not healthy.

14. The method of claim 13 wherein cell health is determined by determining the level of a marker of apoptosis.

15. The method of claim 14 wherein eliminating cells comprises eliminating cells whose level of the marker is above a threshold level.

16. The method of claim 14 wherein the marker comprises cPARP.

17. The method of claim 1 further comprising contacting the immune cells with an inhibitor of one or more T cell subpopulations.

18. The method of claim 17 wherein the inhibitor comprises an inhibitor of Th2 cell subpopulations.

19. The method of claim 17 wherein the inhibitor comprises an inhibitor of Th1 cell subpopulations.

20. The method of claim 17 wherein the inhibitor comprises anti-IL4, anti-IFNg, or a combination thereof.

21. The method of claim 1 wherein the frequency of Th17 cells is determined by determining intracellular levels of one or more markers in single cells

22. The method of claim 21 wherein the intracellular marker comprises IL-17A, IL-17F, or IL-17AF, or a combination thereof.

23. The method of claim 1 wherein the immune cells are from a blood sample or a blood-derived sample, or a tumor infiltrating lymphocyte (TILS) or TILS-derived sample.

24. The method of claim 23 wherein the sample is a peripheral blood mononuclear cell sample.

25. The method of claim 1 further comprising treating the cells with a cytokine.

26. The method of claim 25 wherein the cytokine comprises TNFa, TGFb, IL-1b, IL-21, 11-6 or IL-23, or a combination thereof.

27. The method of claim 26 wherein the cytokine comprises IL-6 or IL-23 or a combination thereof.

28. The method of claim 1 wherein the levels of TNFa and/or IFNg are also determined in single cells.

29. The method of claim 20 wherein the determination of IL-17 levels is performed by flow cytometry or mass cytometry.

30. The method of claim 20 wherein the cells are contacted with detectable binding elements specific for IL-17.

31. The method of claim 1 further comprising determining the level of one or more cell surface markers on the single cells.

32. The method of claim 31 wherein the one or more cell surface markers comprise CD3, CD4, CD8, or a combination thereof.

33. The method of claim 1 further comprising determining the level of IL-21, IL-22, or both, in the cells.

34. A method comprising
(i) contacting immune cells in a culture derived from a sample with one or more activators of T cells and one or more activators of a non-T cell population;
(ii) incubating the cells;
(iii) determining the frequency of Th17 cells in the sample after the incubation.

35. The method of claim 34 wherein the activator of the non-T cell population comprises a toll-like receptor (TLR) activator.

36. A method comprising
(i) contacting immune cells in a culture derived from a sample with one or more activators of T cells;
(ii) incubating the cells;
(iii) determining the level of cell health for single cells of the immune cells and eliminating unhealthy cells from analysis;
(iv) determining the frequency of Th17 cells in the sample after the incubation and after eliminating unhealthy cells.

37. A kit comprising
(i) an activator of T cells;
(ii) a modulator of a non-T cell population;
(iii) a detectable binding element specific for IL-17;
(iv) an agent for inducing IL-17 formation.
B0. The kit of claim 37 wherein the modulator of a non-T cell population comprises a toll-like receptor (TLR) activator.

38. The kit of claim 37 further comprising one or more detectable binding elements to a cell surface marker selected from the group consisting of CD3, CD4, CD8, and combinations thereof.

39. The kit of claim 37 wherein the activator of T cells is a TCR activator.

40. The kit of claim 39 wherein the TCR activator comprises anti-CD3, anti-CD28, or a combination thereof.

41. The kit of claim 37 wherein the TLR activator comprises an activator of TLR4, an activator of TLR 7/8, or a combination thereof.

42. The kit of claim 41 comprising an activator of TLR4 comprising lipopolysaccharide (LPS).

43. The kit of claim 41 comprising an activator of TLR7/8 comprising R848.

44. The kit of claim 37 further comprising a detectable binding element for a marker of cell health.

45. The kit of claim 44 wherein the marker of cell health comprises a marker of apoptosis.

46. The kit of claim 45 wherein the marker of apoptosis comprises cPARP.

47. The kit of claim 37 further comprising instructions.

48. The kit of claim 37 further comprising packaging to hold components of the kit.

49. A kit comprising
(i) an activator of T cells;
(ii) a detectable binding element for a marker of apoptosis;
(iii) a detectable binding element specific for IL-17;
(iv) an agent for inducing IL-17 formation.

50. The kit of claim 49 wherein the marker of apoptosis comprises cleaved PARP.

51. A method of monitoring an aspect of a condition in an individual, comprising
(i) contacting a sample from the individual comprising immune cells with an activator of differentiation of Th17 cells;
(ii) incubating the cells for a period of time;
(iii) determining the change in frequency of Th17 cells in the sample [or determining levels of IL-17 in the cells]; and
(iv) from the results of (iii), determining a characteristic of the aspect of the condition in the individual.

52. The method of claim 51 wherein the individual suffers from an autoimmune condition.

53. The method of claim 52 wherein the immune condition comprises multiple sclerosis, systemic lupus erythematosis, or rheumatoid arthritis.

54. The method of claim 53 wherein the autoimmune condition comprises rheumatoid arthritis (RA).

55. The method of claim 51 wherein the individual suffers from cancer.

56. The method of claim 55 wherein the cancer comprises melanoma, non-small cell lung carcinoma, small cell lung cancer, bladder cancer, or prostate cancer.

57. The method of claim 51 wherein the aspect of the condition is treatment of the condition.

58. The method of claim 57 wherein the condition is cancer and the treatment is treatment for the cancer.

59. The method of claim 58 wherein the treatment comprises an immunomodulatory treatment.

60. The method of claim 59 wherein the immunomodulatory treatment comprises treatment with a checkpoint inhibitor.

61. The method of claim 60 wherein the checkpoint inhibitor comprises ipilimumab.

62. The method of claim 51 wherein the characteristic of the treatment comprises development of adverse effect; progression, regression, or stasis of the condition; response to a treatment; or a combination thereof.

63. The method of claim 62 wherein the characteristic is an adverse effect.

64. The method of claim 64 wherein the adverse effect is the development of colitis.

65. The method of claim 51 wherein the condition is an autoimmune condition and the treatment is a treatment for the autoimmune condition.

66. The method of claim 51 wherein the autoimmune condition comprises multiple sclerosis, systemic lupus erythematosis, or rheumatoid arthritis (RA).

67. The method of claim 66 wherein the condition comprises RA.

68. A method of predicting development of colitis in an individual receiving treatment comprising administration of ipilimumab or potentially comprising administration of ipilimumab comprising
(i) contacting immune cells from a sample from the individual with an activator of Th17 cell differentiation;
(ii) incubating the cells for a period of time;
(iii) determining the change in frequency of Th17 cells after the incubation; and
(iv) from the results of (iii), determining whether or not, or the likelihood, that the individual will develop colitis if the administration of ipilimumab is continued or undertaken.

69. The method of claim 68 wherein the individual is receiving ipilimumab and the method further comprises modifying the treatment of the individual based at least in part on the determination of (iv).

70. The method of claim 69 wherein the modification comprises modifying the dose of ipi, modifying the schedule of dosing of ipilimumab, discontinuing ipilimumab, adding an agent to the treatment, or a combination thereof.

71. The method of claim 68 wherein the individual is potentially receiving ipilimumab and the method comprises administering or not administering ipilimumab based at least in part on the determination of (iv).

72. The method of claim 68 wherein the individual suffers from cancer.

73. The method of claim 72 wherein the cancer comprises melanoma, small cell lung cancer, non-small cell lung carcinoma, bladder cancer, or prostate cancer.

74. The method of claim 73 wherein the cancer comprises melanoma.

75. The method of claim 68 further comprising determining the levels of one or more intracellular activatable elements in immune cells in the sample.

76. The method of claim 75 wherein the cells have further been contacted with a modulator that is not an activator of Th17 cell differentiation.

77. A method of predicting development of colitis in an individual receiving treatment comprising administration of ipilimumab or potentially comprising administration of ipilimumab comprising
(i) determining the levels of one or more activatable elements in immune cells in a sample from the individual; and
(ii) from the results of (i), determining whether or not, or the likelihood, that the individual will develop colitis if the administration of ipi is continued or undertaken.

78. The method of 77 further comprising contacting the cells with a modulator.

79. The method of 77 wherein the activatable element is selected from the group consisting of pStat 1, p-Stat 3, p-Stat 6, p-p38, pERK, IKba, p-S6, p-TBK, p-AKT, IKBa, or a combination thereof.

80. The method of claim 78 wherein the modulator is selected from the group consisting of IL-6, IL-23, R848, IL-1b, LPS, or a combination thereof.

81. The method of claim 80 wherein the modulator comprises IL-6, IL-23, or a combination thereof, and the activatable element comprises p-Stat 1, p-Stat 3, p-Stat 6, or a combination thereof.

82. The method of claim 80, wherein the modulator comprises R848, IL-1b, or a combination thereof, and the activatable element comprises p-p38, pERK, IKba, p-S6, p-TBK, p-AKT, or a combination thereof.

83. The method of claim 80 wherein the modulator comprises LPS and the activatable element comprises p-p38, IKba, p-S6, or a combination thereof.

84. A method of screening agents comprising
(i) contacting immune cells with an activator of Th17 differentiation;
(ii) contacting the cells with an agent;
(iii) incubating the cells for a period of time; and
(iv) determining the frequency of Th17 cells after the incubation.

85. The method of claim 84 further comprising determining whether or not to advance the agent to a further level of screening or tests based at least in part on the results of (iv).

86. The method of claim 84 wherein the agents are screened for potential efficacy in treating one or more conditions.

87. The method of claim 84 wherein the agents are screened for potential adverse effects.

88. The method of claim 86 wherein the condition comprises an autoimmune condition.

89. The method of claim 88 wherein the autoimmune condition comprises multiple sclerosis, systemic lupus erythematosis, or rheumatoid arthritis (RA).

90. The method of claim 84 wherein the agent is an anti-cytokine agent, e.g., an anti-cytokine antibody.
